(19) United States
(12) Patent Application Publication
(10) Pub. No.: US 2017/0008886 A1
(43) Pub. Date: Jan. 12, 2017

(54) A PROCESS FOR THE PREPARATION OF APIXABAN AND ITS INTERMEDIATES

(71) Applicant: WANBURY LTD., Navi Mumbai, Maharashtra (IN)

(72) Inventors: Nitin Sharadchandra Pradhan, Navi Mumbai (IN); SACHIN ULHAS SONAVANE, Navi Mumbai (IN); DAYAGHAN GHANGADHAR PATIL, Navi Mumbai (IN); UTTAM SAKHARAM PUJARI, Navi Mumbai (IN); RAVINDRA BHAUSAHEB PAGIRE, Navi Mumbai (IN)

(73) Assignee: Wanbury Ltd., Navi Mumbai Mahrashtra (IN)

(21) Appl. No.: 15/113,430

(22) PCT Filed: Jan. 6, 2015
(86) PCT No.: PCT/IN2015/000007

§ 371 Date: Jul. 21, 2016

(30) Foreign Application Priority Data
Jan. 21, 2014 (IN) 197/MUM/2014

(51) Int. Cl. C07D 471/04

(52) U.S. Cl. CPC C07D 471/04

(57) ABSTRACT

The present invention discloses the novel process for preparation of Apixaban intermediate formula (D), intermediate formula (E) and preparation of Apixaban from theses intermediates.

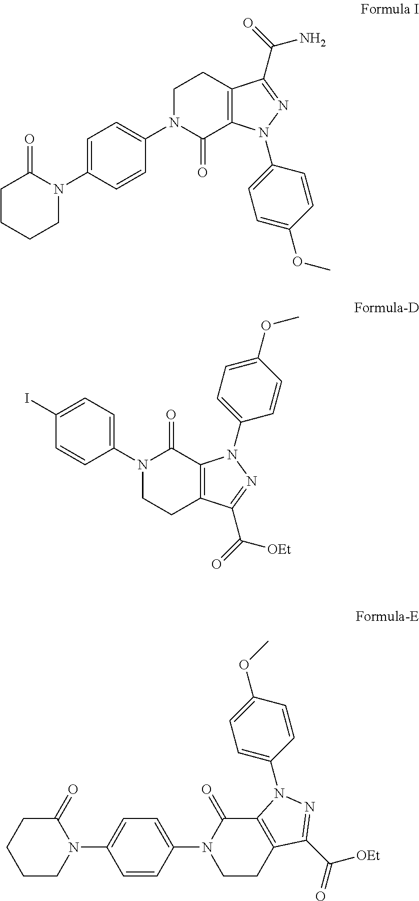 [SMILES: COc1ccc(-n2nc(C(N)=O)c3c2C(=O)N(c2ccc(N4CCCCC4=O)cc2)CC3)cc1 ; CCOC(=O)c1nn(-c2ccc(OC)cc2)c2c1CCN(c1ccc(I)cc1)C2=O ; CCOC(=O)c1nn(-c2ccc(OC)cc2)c2c1CCN(c1ccc(N3CCCCC3=O)cc1)C2=O]

### FIELD OF THE INVENTION

The present invention mainly relates to the process for preparation of Apixaban (formula-I) or pharmaceutically accepted salts or solvates or hydrate form. This instant invention further relates to process for preparation of Apixaban intermediates, namely ethyl 6-(4-iodophenyl)-1-(4-methoxyphenyl)-7-oxo-4,5,6,7-tetrahydro-1H-pyrazolo[3,4-c]pyridine-3-carboxylate (Formula-D) and 6-(4-pyridinone)-1-(4-methoxyphenyl)-7-oxo-4,5,6,7-tetrahydro-1H-pyrazolo[3,4-c]pyridine-3-carboxylate (Formula-E). The formula (I), formula (D) and formula (E) are structurally represented as below;

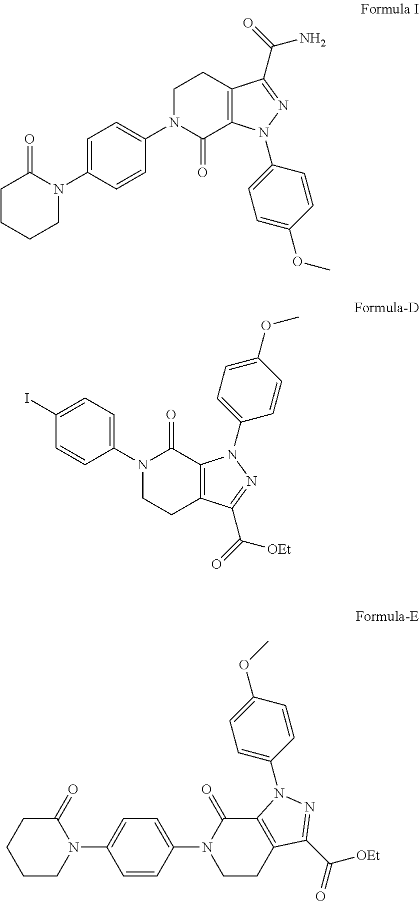 [SMILES: COc1ccc(-n2nc(C(N)=O)c3c2C(=O)N(c2ccc(N4CCCCC4=O)cc2)CC3)cc1 ; CCOC(=O)c1nn(-c2ccc(OC)cc2)c2c1CCN(c1ccc(I)cc1)C2=O ; CCOC(=O)c1nn(-c2ccc(OC)cc2)c2c1CCN(c1ccc(N3CCCCC3=O)cc1)C2=O]

### BACKGROUND OF THE INVENTION

“Apixaban” is chemically known as 4,5,6,7-tetrahydro-1-(4-methoxyphenyl)-7-oxo-6-[4-(2-oxo-1-piperidinyl)phenyl]-1H-pyrazolo[3,4-c]pyridine-3-carboxamide or 1-(4-methoxyphenyl)-7-oxo-6-[4-(2-oxo-1-piperidinyl)phenyl]-4,5,6,7-tetrahydro-1H-pyrazolo [3,4-c]pyridine-3-carboxamide (IUPAC name) of Formula (I).

Apixaban is highly potent, selective, and orally bioavailable inhibitor of blood coagulation factor Xa (fXa), was developed in a late-stage clinic trial for the prevention and treatment of thromboembolic diseases by Bristol-Myers Squibb. [Thromb. Haemost. 2010, 104, 301-310 and J. cardiovasc. Pharm. 2010, 55, 609-616] It could be marketed for the treatment of deep vein thrombosis (DVT) and venous thrombosis as a new-generation anticoagulant. [J. Thromb. Haemost. 2008, 6, 1313-1318] Moreover, it has also shown promise in treating acute coronary syndrome (ACS), [Thromb. Haemost. 2010, 104, 976-983] cerebrovascular ischemia, and cancer. [Arterioscler. Thromb. Vasc. Biol. 2007, 27, 1238-1247].

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Apixaban firstly disclosed in U.S. Pat. No. 6,967,208 wherein, Apixaban has indicated first as its requirement for the use as an antithrombotic agent and thus being developed for oral administration.

U.S. Pat. No. 7,153,960 discloses the process for preparation of Apixaban Wherein the formula-D (Scheme-1) is prepared by the cycloaddition reaction of formula(A), and formula(B), to form formula(C), which is in-situ converted to formula (D), by treatment with acid in specifically aprotic solvent. Further formula (D), is subsequently converted to formula (E), by Ullmann coupling reaction. Process for the preparation of formula (D) & (E), are complex, tedious, time consuming and involves too many operations in order to isolate these intermediates, formula (D) & (E), and also requires purification to obtain pure compound.

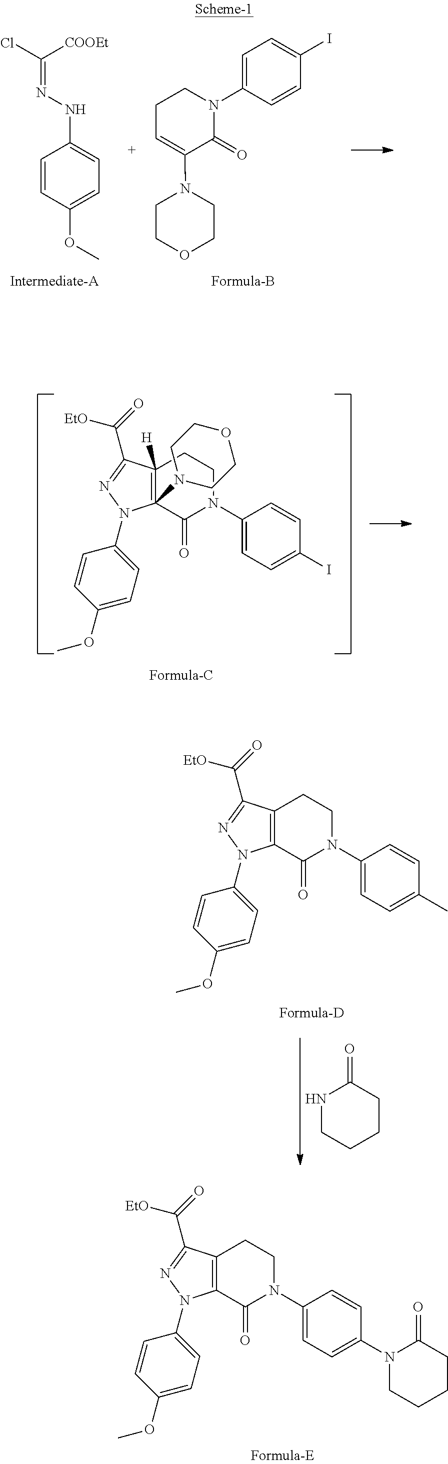

Several routes for the preparation of apixaban have been reported; J. Med. Chem. 2007, 50, 5339-5356 reported a similar strategy for the synthesis of formula (I). Out of this one route is as scheme mentioned in scheme-1, however, only 21% yield was obtained in the key Ullmann coupling reaction of cycloadduct formula (D) with d-Valerolactum to obtain formula (E).

WO-2010/030983, disclosed a similar pathway for synthesis of formula [I], as described in scheme-1, with marginal increase in yield of the Ullmann reaction ˜29% yield is reported.

WO-2012/168364, discloses the preparation of formula(E), (Ullmann coupling reaction) by using the base as K₃PO₄ and N′N′ dimethyl ethylenediamine in toluene as a solvent with slight improved in yield up to 67%, after the crystallization of crude in ethyl acetate.

WO2003/049681, discloses alternate methodology, which underwent an Ullmann coupling with iodide in the presence of cuprous iodide to obtained intermediate(C), in 68% yield. The Same instant application also teaches the requirement of expensive organic cuprous compound Cu(PPh₃)₃Br as catalyst for the Ullmann coupling reaction in order to enhanced yield up to 68%.

The complexity of the known processes for the preparation of Formula (D) & (E), uses reagents are expensive and requires drastic reaction conditions and long reaction time so, the said process are very intricate to apply for large scale preparation, especially for the purpose of preparing intermediates of formula (D) and formula(E). So ultimately these synthetic methods cannot meet the demand of a large-scale preparation of formula (I), in terms of cost, process simplicity and enhanced productivity.

As part of the processes of bringing a new cost effective process for active pharmaceutical ingredient (API) to market, it often requires use of an alternative synthetic strategy which concomitantly exerted cost significant with environment friendly. So in order to overcome theses said drawbacks, We involved the novel concept for the preparation of formula (I), wherein, the method exert to achieve respectful yield of formula(E), penultimate stage to form Apixaban formula (I), by using effective reaction combo pack such as intermediate (D)treated with valerolactum in toluene as a solvent, cuprous iodide (Cup as a catalyst, cesium carbonate as base and di-methyl aniline (DMA) as Ligand precursor for the Ullmann coupling reaction to furnish the formula(E) in 88-90% yield. Ullmann coupling reactions which is key stage for reduced overall yield described in above prior arts. Therefore, the strategy has displayed its practical application potential.

Further we surprisingly found that the use of precursor dimethylaniline and base cesium carbonate along with cuprous iodide (CuI) as catalyst played a vital role in reaction which is mainly responsible to enhance yield (85-90%) with shortened reaction time and good reproducibility as well.

### SUMMARY OF THE INVENTION

In its main aspect the present invention is to provide the novel process for preparation of compound formula (D), by treating compound formula (A), with compound formula (B), in polar protic or aprotic solvents in presence of base to obtained formula (c), which concomitantly treated with suitable acid in polar solvent to obtained formula (D).

In another aspect the present invention is to provide the novel process for preparation of compound formula (E), wherein intermediate formula (D), is treated with valerolactum in aprotic solvents in presence of suitable base, ligand and catalyst to exert intermediate formula (E).

In yet another aspect present invention is to provide the novel process for preparation of Apixaban of compound formula (I), by amidation of compound formula (E), with suitable aminating agent in polar solvent or without polar solvent.

In yet another aspect present invention is to provide the compound formula (I); Apixaban which is substantially free from the impurity of compound formula (F), by dissolving Apixaban in suitable solvent and extracted with carbonate water solution.

In yet another aspect present invention is to provide the compound formula (I); Apixaban which is substantially free from the impurity of compound formula (G), by crystallizing the formula (I), with suitable polar solvents mixture.

### DETAILED DESCRIPTION OF THE INVENTION

The main aspect of the present invention is to introduced novel process for the preparation of intermediate (D) & intermediate (E), and process for Apixaban formula (I). Further the present invention introduced the novel reaction combi-pack for Ullmann coupling which accountable to enhance the yield of formula (E), and concomitantly exert Apixaban of formula [I].

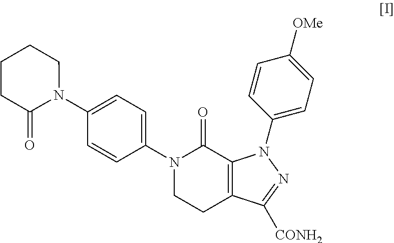 [SMILES: COc1ccc(-n2nc(C(N)=O)c3c2C(=O)N(c2ccc(N4CCCCC4=O)cc2)CC3)cc1]

In a first embodiment, the present invention provides an novel process for preparing an intermediate (D), wherein reacting intermediate (A), and intermediate (B), in presence of organic base selected from triethylamine (TEA), Diisopropyl ethyalamine and in protic polar or aprotic non polar solvents selected from methanol,ethanol,iso-Propanol,n-propanol,iso-amyl alcohol,butanol and toluene or mixture thereof to form cycloaddition product of an intermediate formula(C).

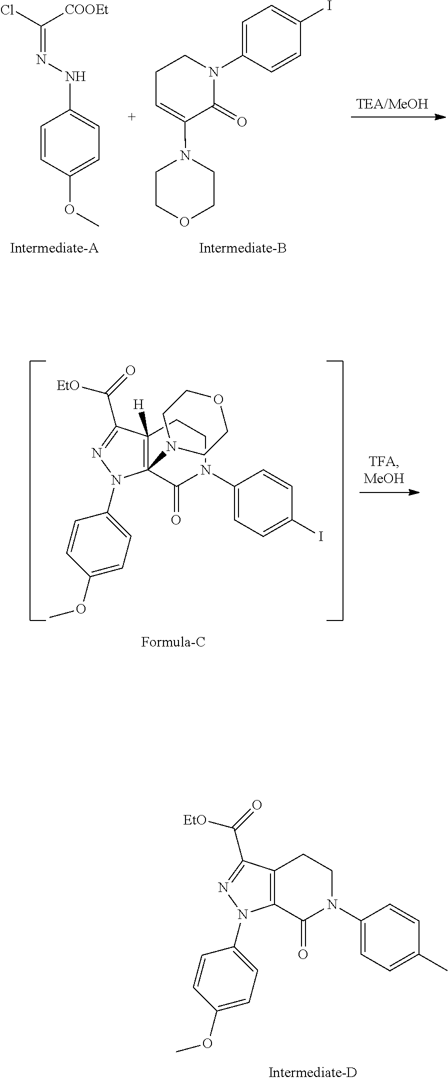

Intermediate(C), is further in situ reacted with acid such as trifluoroacetic acid (TFA), acetic acid, sulphuric acid, and Hydrochloric acid or mixture thereof in polar protic solvents selected from methanol, ethanol iso-Propanol, n-propanol, iso-amyl alcohol and butanol or mixture thereof to obtain intermediate(D), in pure state and high yield without any further purification.

The solvent used for the preparation of intermediate (D) is preferably a methanol as methanol exert the good yield and the after completion of reaction solid come up in the same reaction mass without addition of any solvent and without any work up of reaction. The obtained solid having the good purity without any further purification.

The added advantage to use of methanol as solvent is that, methanol is used as a solvent for reaction and isolation as well by avoiding the number of solvents usage and without work up of reaction which concomitantly exert environment friendly with economic significance.

In a second embodiment, the present invention provides an novel process for preparing an intermediate (E), wherein reacting intermediate (D), with piperidine-2-one (valarolactum) in toluene as a solvent and in presence of base selected from cesium carbonate,potassium carbonate and potassium terbutoxide with ligand precursor selected from dimethylaniline (DMA), Dimethyl aminopyridine(DMAP) and 8-hyrdoxyquinoline, with catalytic amount of cuprous iodide (CuI) to obtain intermediate (E), in pure form with high yield without any further purification (yield ˜85-90%). The unique or selective combination of reaction pack is cesium carbonate as a base, dimethylaniline (DMA) as ligand precursor and catalyst CuI which are mainly accountable for enhancement in yield without undergoing any side reactions.

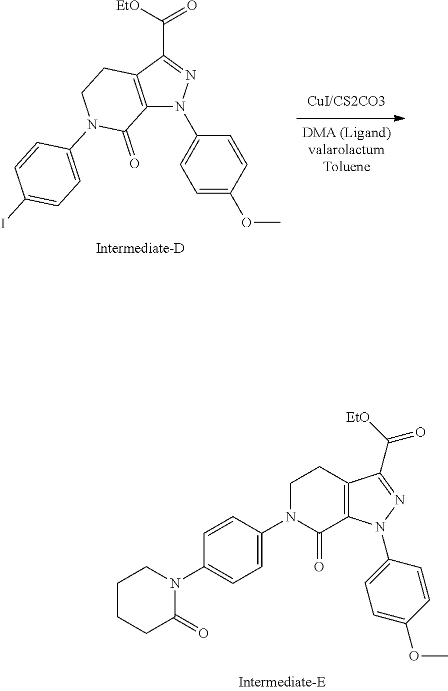 [SMILES: CCOC(=O)c1nn(-c2ccc(OC)cc2)c2c1CCN(c1ccc(I)cc1)C2=O ; CCOC(=O)c1nn(-c2ccc(OC)cc2)c2c1CCN(c1ccc(N3CCCCC3=O)cc1)C2=O]

In a third embodiment, the present invention provides a process for preparing Apixaban [I], by amidation reaction using aqueous ammonia or mixture of aqueous ammonia in polar protic solvents selected from methanol, ethanol, isopropanol, n-propanol, iso-amyl alcohol and butanol or mixture thereof at 65-70° C. for 4-8, hours to obtain apixaban containing acid impurity of formula (F) in ˜1-2%.

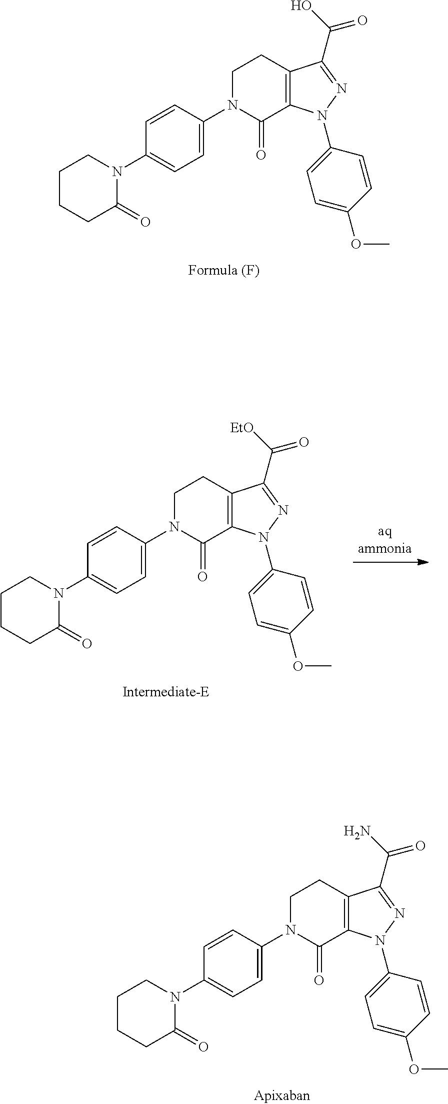 [SMILES: COc1ccc(-n2nc(C(=O)O)c3c2C(=O)N(c2ccc(N4CCCCC4=O)cc2)CC3)cc1 ; CCOC(=O)c1nn(-c2ccc(OC)cc2)c2c1CCN(c1ccc(N3CCCCC3=O)cc1)C2=O ; COc1ccc(-n2nc(C(N)=O)c3c2C(=O)N(c2ccc(N4CCCCC4=O)cc2)CC3)cc1]

The obtained Apixaban with acid impurity is dissolved water immiscible solvents selected from ethyl acetate, methylene dichloride and ethylene dichloride or mixture thereof and washed with 2-5% sodium bicarbonate solution further evaporated the water immiscible solvents under reduced pressure followed by crystallization in mixture of methanol-water to obtain pure Apixaban [I] without any further purification with high yield and purity as per the ICH guideline.

The organic layer obtained by dissolving Apixaban containing acid impurity in water immiscible solvents is washed by sodium bicarbonate solution in order to get the substantially free from the impurity of compound formula (F). Finally obtained Apixaban free from compound formula (F), crystallized in protic solvent selected from methanol, water, ethanol, n-propanol, iso-propanol and butanol or mixture thereof, to get the pure Apixaban as per the ICH guideline, wherein the crystallization carried out to minimize the impurity of compound formula (G), a trans-esterification impurity formed during the amidation reaction.

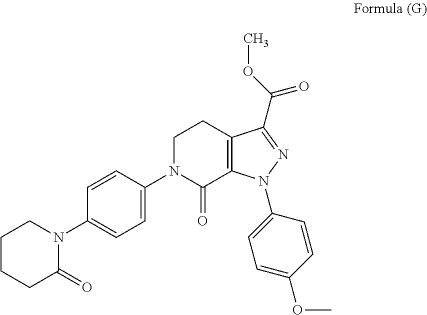 [SMILES: COC(=O)c1nn(-c2ccc(OC)cc2)c2c1CCN(c1ccc(N3CCCCC3=O)cc1)C2=O]

The following examples illustrate the invention

### Abbreviations and Acronyms:

HPLC: High performance liquid chromatography

TFA: Tri-Fluro acetic acid

TEA: Tri-ethylamine

MDC: Methylene dichloride

### EXAMPLES

### Example-1

### Synthesis of Intermediate Formula D: ethyl 6-(4-iodophenyI)-1-(4-methoxyphenyl)-7-oxo-4,5,6,7-tetrahydro-1H-pyrazolo [3,4-c] pyridine-3-carboxylate

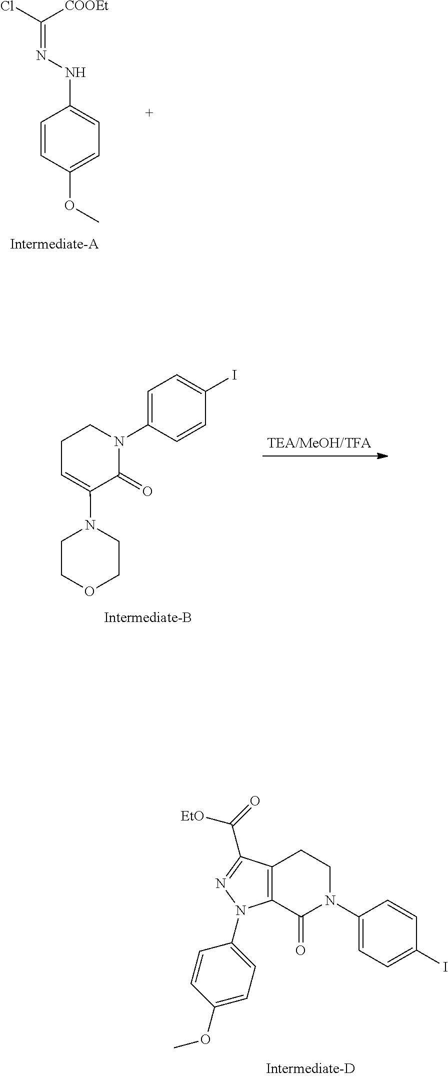 [SMILES: CCOC(=O)/C(Cl)=N\Nc1ccc(OC)cc1 ; O=C1C(N2CCOCC2)=CCCN1c1ccc(I)cc1 ; CCOC(=O)c1nn(-c2ccc(OC)cc2)c2c1CCN(c1ccc(I)cc1)C2=O]

Intermediate-B (25 g, 0.065 moles) toluene (250 ml), Intermediate-A (20.68 g, 0.08 moles)and TEA (10 g, 0.099 moles); were heated to reflux for 2-3 hrs progress of the reaction is monitored on TLC or HPLC. Distilled out toluene from reaction mass under reduced pressure below 70° C. to obtain thick brown mass. To this add 400 ml methanol, cooled to 25-30° C. and slowly add TFA (15 g, 0.135 moles). Reaction mass was then stirred at RT for ˜1.0-1.5 hr reaction monitored on TLC or HPLC. Solid was isolated by filtration flush with fresh 25 ml methanol. Dried at 60-65° C. to obtain Intermediate-D 29.6 gm (88% yield) and HPLC purity >99% without any purification.

### Example-2

### Synthesis of Compound of Formula D: ethyl 6-(4-iodophenyI)-1-(4-methoxyphenyl)-7-oxo-4,5,6,7-tetrahydro-1H-pyrazolo [3,4-c] pyridine-3-carboxylate

Intermediate-B (25 g, 0.065 moles), toluene (250 ml), intermediate-A (20.68 g, 0.08 moles) and TEA (10 g, 0.099 moles) were heated to reflux for 2-3 hrs completion of reaction is checked on TLC or HPLC. Toluene from reaction mass was then distilled out under reduced pressure to obtain thick brown mass. To this add 400 ml methanol, cooled to 25-30° C. and add drop wise 6.0 gm conc. Sulphuric acid. Reaction mass is stirred at RT for ˜1.0-1.5 hr to obtain solid which is filter and washed with fresh 25 ml methanol. Dried at 60-65° C. to obtain Intermediate-D 29.6 gm (88-90% yield) and HPLC purity >99% without any purification.

### Example-3

### Synthesis of Compound of Formula D: ethyl 6-(4-iodophenyl)-1-(4-methoxyphenyl)-7-oxo-4,5,6,7-tetrahydro-1H-pyrazolo [3,4-c] pyridine-3-carboxylate

Intermediate-B (0.065 moles), toluene (250 ml), Intermediate-A (0.08 moles) and TEA (0.099 moles); were heated to reflux for 2-3 hrs progress of the reaction is monitored on TLC or HPLC. Solvent switched to methanol and cooled to 25-30° C. To the above reaction mass add drop wise 10.0 gm glacial acetic acid. Reaction mass is stirred at RT for ˜5.0 hrs and completion of the reaction is checked on TLC or HPLC. Solid is isolated by filtration, washed with fresh 25 ml methanol. Dried at 60-65° C. to obtain Intermediate-D 27.6 gm (82% yield) and HPLC purity >99% without any purification.

### Example-4

### Synthesis of Compound of Formula E: 1-(4-methoxyphenyI)-7-oxo-6-(4-(2-oxopiperidin-1-yl)phenyl)-4,5,6,7-tetrahydro-1H-pyrazolo [3,4-c] pyridine-3-carboxalic acid ethyl ester.

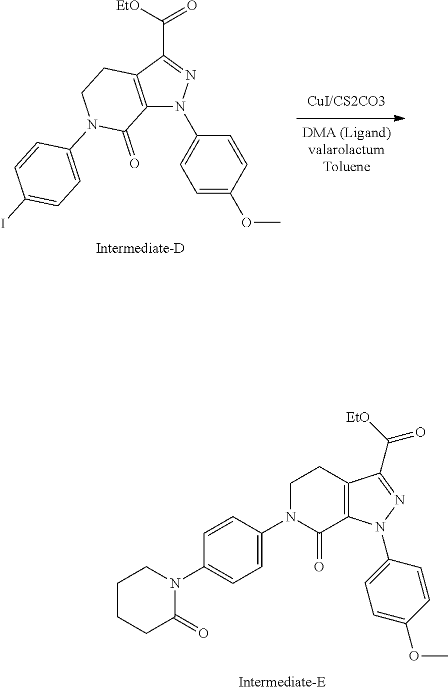 [SMILES: CCOC(=O)c1nn(-c2ccc(OC)cc2)c2c1CCN(c1ccc(I)cc1)C2=O ; CCOC(=O)c1nn(-c2ccc(OC)cc2)c2c1CCN(c1ccc(N3CCCCC3=O)cc1)C2=O]

Charge toluene 1900 ml, deoxygenate by purging nitrogen under vigorous stirring. Charge Intermediate-D (110 g, 0.212 moles) valarolactum (64 gm, 0.645 moles) cesium carbonate (83.5 gm, 0.256 moles). To the above reaction mass charge CuI (8.1 gm, 0.042 moles) and Di-Methyl aniline (DMA) (3.0 gm, 0.024 moles) under nitrogen atmosphere. Heat and maintain the reaction mass under stirring at 110° C. Monitor the progress of the reaction by HPLC. Cool the reaction mass under stirring and filter through Hyflo bed and flush the flask and bed with toluene 2*110 ml. wash the clear filtrate with 5% aqueous HCl solution. Toluene layer is concentrated to ˜70% of its volume under reduced pressure. lntermediate-E gets precipitated out as a crystalline material with very high yield ˜88% and purity >99%.

### Example-5

### Synthesis of Compound of Formula E: 1-(4-methoxyphenyl)-7-oxo-6-(4-(2-oxopiperidin-1-yl)phenyl)-4,5,6,7-tetrahydro-1H-pyrazolo [3,4-c] pyridine-3-carboxalic acid ethyl ester.

By following the process as described in example-4 and employed the different ligand precursor the results obtained are reflected below:\

| Ligand precursor | yield | purity by HPLC |
|---|---|---|
| Di-methyl amino pyridine | 75% | >99% |
| 8-Hydroxyquinoline | 64% | >99% |

### Example-6

### Synthesis of Compound of Formula E: 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxalic acid ethyl ester

By following the process as described in example-4 and employed, the different base the results obtained are reflected below:

| Base | yield | purity by HPLC |
|---|---|---|
| Potassium carbonate | 80% | >99% |
| Potassium ter-butoxide | 74% | >99% |

### Example 7

### Synthesis of Compound of Formula [I]: 4,5,6,7-tetrahydro-1-(4-methoxyphenyl)-7-oxo-6-[4-(2-oxo-1-piperidinyl) phenyl]-1H-pyrazolo [3,4-c] pyridine-3-carboxamide (Crude Apixaban)

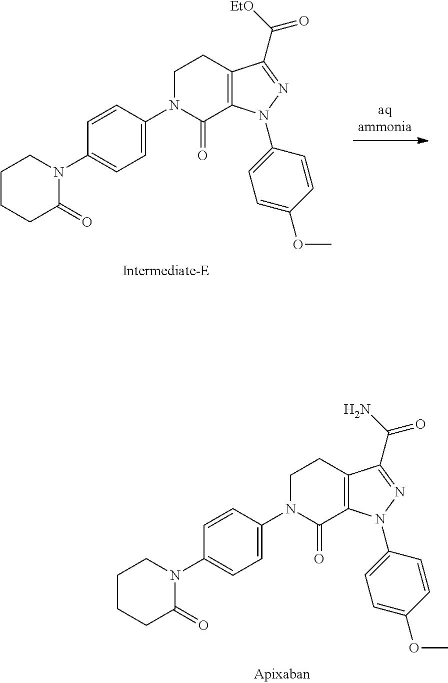 [SMILES: CCOC(=O)c1nn(-c2ccc(OC)cc2)c2c1CCN(c1ccc(N3CCCCC3=O)cc1)C2=O ; COc1ccc(-n2nc(C(N)=O)c3c2C(=O)N(c2ccc(N4CCCCC4=O)cc2)CC3)cc1]

Intermediate-E (65 g, 0.133 moles) was suspended in 30% aq. ammonia solution in methanol. Mixture was heated to ˜70° C. in an autoclave. Aqueous ammonia develops in-build pressure of ˜1-2 kg. Progress of the reaction is monitored on TLC/HPLC. Reaction gets completed in 5-10 hrs, cooled to RT and methanol is distilled out under reduced pressure. To the reaction mass add water and product was filtered and washed with water and dried at 60-65 ° C. to obtain crude Apixaban 46.9 g with HPLC purity ˜98.5%. Crude apixaban contains an acid impurity ˜1.0-1.5%. It is further dissolved in methylene dichloride (MDC) and washed with 2.0-5.0% bicarbonate solution; MDC layer is then concentrated under reduced pressure and crystallize using mixture of solvents methanol water to obtain pure apixaban with HPLC purity >99%.

### Example 8

### Synthesis of Compound of Formula [I]: 4,5,6,7-tetrahydro-1-(4-methoxyphenyl)-7-oxo-6-[4-(2-oxo-1-piperidinyl) phenyl]-1H-pyrazolo [3,4-c] pyridine-3-carboxamide (Crude Apixaban)

By following the process as described in example-7 and employed the different solvent with aq. ammonia the results obtained are reflected below:

| Solvents | Yield (%) | purity by HPLC |
|---|---|---|
| n-propanol & Iso-propanol | 69 | >99% |
| butanol | 63 | >99% |
| Ethnol | 69 | |
| Iso-amyl alcohol | 62 | |

### Example-9

### Synthesis of Compound of Formula [I]: 4,5,6,7-tetrahydro-1-(4-methoxyphenyl)-7-oxo-6-[4-(2-oxo-1-piperidinyl) phenyl]-1H-pyrazolo [3,4-c] pyridine-3-carboxamide (Apixaban)

Intermediate-E (35 g, 0.0716 moles) is suspended in 30% aq. ammonia solution. Mixture was heated to ˜80-90° C. in an autoclave. Aqueous ammonia develops in-build pressure of ˜1-2 kg. Progress of the reaction is monitored on TLC/HPLC. When reaction was completed in ˜10 hrs, reaction mass is cooled to RT and diluted with water. Product is filter and washed with water, dried at 60-65 ° C. to obtain crude apixaban 23-25 gm with HPLC purity ˜98.5%. This crude product contains acid impurity approx-1.0-1.5%. Crude apixaban is further dissolved in MDC and washed with 2.0-5.0% bicarbonate solution. MDC layer is then concentrated under reduced pressure and crystallize using mixture of solvents methanol water to obtain pure apixaban with HPLC purity >99% (yield 61%).

The crystallization of Apixaban has been carried out in different solvents with water the results obtained are depicted in tables as below:

| | | purity by |
|---|---|---|
| Solvents for crystallization | Yield (%) | HPLC |
| Water-ethanol | 58 | >99.5% |
| Water-iso-propanol | 55 | >99.5% |
| Water-butanol | 51 | >99.5% |
| Water-n-propanol | 56 | >99.5% |

1. A process for preparation of Apixaban having formula (I): 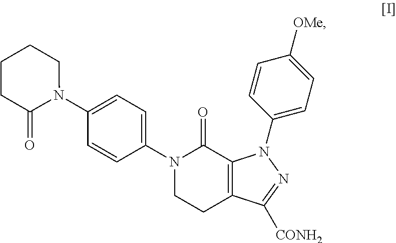 [SMILES: COc1ccc(-n2nc(C(N)=O)c3c2C(=O)N(c2ccc(N4CCCCC4=O)cc2)CC3)cc1]
comprising:
a) reacting an intermediate having formula (A): 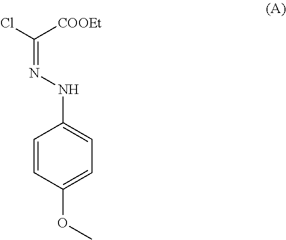 [SMILES: CCOC(=O)/C(Cl)=N\Nc1ccc(OC)cc1]
with an intermediate having formula (B): 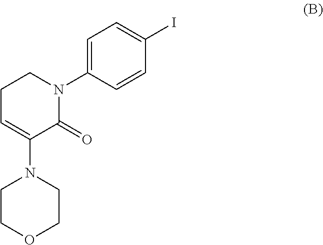 [SMILES: O=C1C(N2CCOCC2)=CCCN1c1ccc(I)cc1]
in a solvent in presence of a base to obtain an intermediate having formula (C): 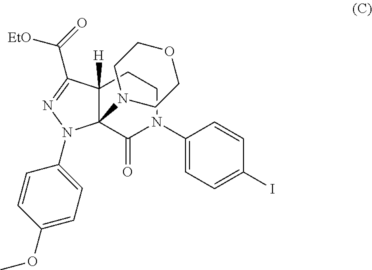 [SMILES: CCOC(=O)C1=NN(c2ccc(OC)cc2)[C@@]2(N3CCOCC3)C(=O)N(c3ccc(I)cc3)CC[C@@H]12]
which is further treated with acid in a polar solvent to obtained an intermediate having formula (D): 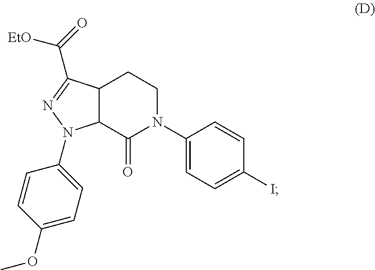 [SMILES: CCOC(=O)C1=NN(c2ccc(OC)cc2)C2C(=O)N(c3ccc(I)cc3)CCC12]
b) treating the intermediate having formula (D) with piperidine-2-one (valarolactum) in a solvent in presence of a base, a ligand precursor and a catalyst, to obtain an intermediate having formula (E): 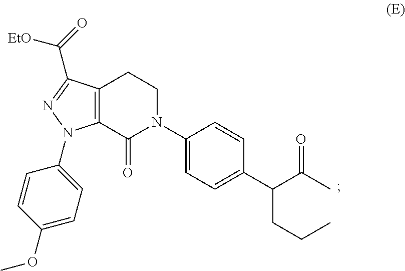 [SMILES: CCCC(C(C)=O)c1ccc(N2CCc3c(C(=O)OCC)nn(-c4ccc(OC)cc4)c3C2=O)cc1]
c) treating the intermediate having formula (E) with aqueous ammonia or aqueous ammonia with a polar protic solvent to obtain crude apixaban;
d) crude apixaban from step (c) is dissolved in a water immiscible solvent and extracting the organic layer using a basic water solution using a suitable base and evaporating the organic layer to obtain apixaban free from a compound having formula (F): 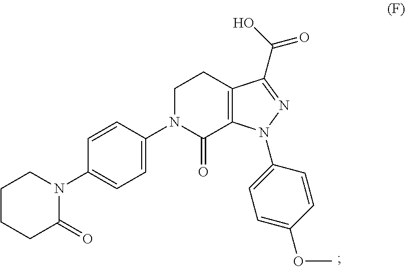 [SMILES: COc1ccc(-n2nc(C(=O)O)c3c2C(=O)N(c2ccc(N4CCCCC4=O)cc2)CC3)cc1]
e) the apixaban obtained from step (d) is crystallized in a polar solvent to obtain pure Apixaban free from a compound having formula (G):

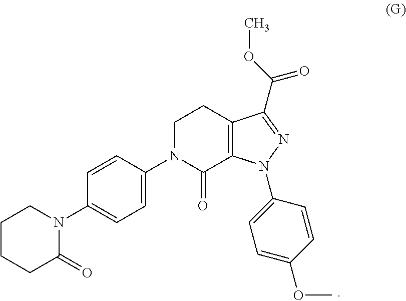 [SMILES: COC(=O)c1nn(-c2ccc(OC)cc2)c2c1CCN(c1ccc(N3CCCCC3=O)cc1)C2=O]

2. The process of claim 1, wherein the solvent of step (a) is methanol and the base of step (a) is triethylamine.

3. The process of claim 1, wherein the solvent of step (b) is toluene; the base of step (b) is selected from a group consisting of cesium carbonate, potassium carbonate and pot-ter-butoxide; the ligand precursor of step (b) is selected from a group consisting of dimethylaniline, dimethylaminopyridine, and 8-hydroxyquinoline and the catalyst of step (b) is cupric iodide.

4. The process of claim 1, wherein the base of step (b) is cesium carbonate.

5. The process of claim 1, wherein the ligand precursor of step (b) is dimethylaniline.

6. The process of claim 1, wherein the polar protic solvent of step (c) is selected from a group consisting of methanol, ethanol, propanol, butanol and isoamyl alcohol, or a mixture thereof.

7. The process of claim 1, wherein the polar protic solvent of step (c) is methanol.

8. The process of claim 1, wherein the water immiscible solvent of step (d) is selected from a group consisting of ethyl acetate, methylene dichloride and ethylene dichloride, or mixture thereof.

9. The process of claim 1, wherein water immiscible solvent of step (d) is methylene dichloride.

10. The process of claim 1, wherein the suitable base of step (d) is selected from a group consisting of sodium bicarbonate and potassium carbonate.

11. The process of claim 1, wherein the polar solvent of step (e) is selected from a group consisting of methanol, ethanol, water, propanol and butanol or mixture thereof.

12. The process of claim 1, wherein the polar solvent of step (e) is a mixture of water and methanol.

13. The process of claim 2, wherein the base of step (b) is cesium carbonate.

14. The process of claim 3, wherein the base of step (b) is cesium carbonate.

15. The process of claim 2, wherein the ligand precursor of step (b) is dimethylaniline.

16. The process of claim 3, wherein the ligand precursor of step (b) is dimethylaniline.
